(19) United States
(12) Patent Application Publication
(10) Pub. No.: US 2003/0166703 A1
(43) Pub. Date: Sep. 4, 2003

(54) Method of treatment

(76) Inventors: Joanne Bright, Marlow Essex (GB); Nick Harrington, Egham Surrey (GB); Naheed Mirza, Ballerup (DK); Kelly Stanhope, Ascot Berks (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/257,175

(22) PCT Filed: Apr. 6, 2001
(86) PCT No.: PCT/EP01/03926

(30) Foreign Application Priority Data
Apr. 11, 2000 (GB) 0008921.9

(51) Int. Cl. A61K 31/407

(52) U.S. Cl. 514/412

(57) ABSTRACT

The use of [R-(Z)]-α-(methoxyimino)-α-(1-azabicyclo[2.2.2]oct-3-yl)acetonitrile or a pharmaceutically acceptable salt thereof for the treatment of anxiety.

This invention relates to a method for the treatment of anxiety and to a compound for use in such method.

EP-A-0392803 (Beecham Group p.l.c.) discloses certain azabicyclic compounds which enhance acetylcholine function via an action at muscarinic receptors within the central nervous system, including [R-(Z)]-α-(methoxyimino)-α-(1-azabicyclo[2.2.2]oct-3-yl) acetonitrile (Compound (I)), and pharmaceutically acceptable salts, their use in the treatment and/or prophylaxis of dementia and processes by which such compounds may be made.

WO-93/17018 and WO-95/31456 disclose alternative processes by which Compound (I) may be made.

WO-00/03717 discloses the use of Compound (I) or a pharmaceutically acceptable salt thereof for the treatment of psychosis or other neuropsychiatric symptoms in patients with Alzheimer's Disease with severe behavioural disturbance.

Certain muscarinic agonists have been claimed to have atypical antipsychotic-like effects in animal models of schizophrenia (e.g., Bymaster et al. European Journal of Pharmacology, 356, 109-19, 1998). Certain atypical, but not typical antipsychotics are claimed to reduce anxiety in both animal models and in schizophrenics (eg., Moore et al., Behav. Pharmacol, 5, 196-202, 1994; Tollefson et al., Biological Psychiatry, 43, 803-810, 1998).

It has now been found that Compound (I) is also of potential use in the treatment of anxiety, more particularly in patients other than those with Alzheimers's Disease.

According to the present invention, there is provided the use of Compound (I) or a pharmaceutically acceptable salt thereof in the manufacture of a medicament for the treatment of anxiety.

In a further aspect the invention provides a method for the treatment of anxiety comprising administering to the patient an effective, non-toxic amount of Compound (I) or a pharmaceutically acceptable salt thereof.

Compound (I) can form acid addition salts with strong acids. The term pharmaceutically acceptable salt encompasses solvates and hydrates.

Compound (I) is preferably provided in a pharmaceutical composition, which comprises Compound (I) or a pharmaceutically acceptable salt thereof, and a pharmaceutically acceptable carrier. The invention thus further provides a pharmaceutical composition, which comprises Compound (I) or a pharmaceutically acceptable salt thereof for use in the treatment of anxiety.

In a preferred aspect Compound (I) is provided in the form of the monohydrochloride.

The composition may be in the form of tablets, capsules, powders, granules, lozenges, suppositories, reconstitutable powders. or liquid preparations such as oral or sterile parenteral solutions or suspensions.

In order to obtain consistency of administration it is preferred that a composition is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

Solid oral compositions may be prepared by conventional methods of blending, filling, tabletting or the like. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, or hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The composition may contain from 0.1% to 99% by weight, preferably from 10-60% by weight, of the active material, depending on the method of administration.

The dose of the compound will vary in the usual way with the seriousness of the disorder, the weight of the sufferer, and the relative efficacy of the compound. However, as a general guide suitable daily doses below 0.01 mg/kg more particularly 0.003 mg/kg and below, for example 0.0001-0.003 mg/kg, such as 0.00035-0.003 mg/kg, 0.0007-0.003 mg/kg, 0.0001-0.0007 mg/kg or 0.00035-0.002 mg/kg. Suitable unit doses to achieve such daily doses are 5, 12.5, 25, 50 or 75 μg, administered twice daily and, in the case of 50 μg, once daily.

Within the above indicated dosage ranges no unacceptable toxicological effects are indicated for Compound (I).

The following pharmacological data illustrates the invention.

Methods

Rat Conditioned Emotional Response (CER) Model of Anxiety

Sixty male Lister Hooded rats with a mean free-feeding weight of 472 g were used. Rats were housed in cages of four and were fed a restricted diet designed to maintain rats at 85% of their free-feeding body weight. A 12:12 light/dark cycle was in effect in the colony room and the rats were trained and tested during the light part of the cycle.

Twenty-four Coulbourn operant chambers (E10-18TC) with associated Coulbourn pellet dispensers (E14-24) and operant levers (E23 -17) were used (Coulbourn Instruments equipment supplied by Bilaney Consultants GmbH, Schirnerstrasse 23, 40211, Dusseldorf, Germany). These chambers were housed in outer sound and light attenuating shells equipped with a ventilation fan which also helped to mask external noise. A single operant lever was positioned on the left side of the front panel of the operant chamber, approximately 2 cm above the grid floor. Coulbourn animal test cage grid floor shockers (E13-14) were used to deliver shock (0.4 mA, 0.5 s) to the grid floors (model E-10-10SF). Formula P Noyes food pellets (45 mg) (supplied by Sandown Scientific, Beards Lodge, 25 Oldfield Road, Hampton, Middlesex. TW 12 2AJ) could be delivered to the food magazine centred on the front wall of the chamber approximately 2 cm above the floor. A houselight (4.0 mA, 28V) was positioned on the front wall of the chamber, above the food magazine, approximately 1.0 cm below the ceiling. The operant chambers were controlled and the number of lever presses recorded by an Acorn computer programmed in Paul Fray Arachnid software (CENES Pharmaceuticals. Compass House, Vision Park, Chivers Way, Histon, Cambridge, CB4 9ZR).

Once the rats had been placed on a restricted feeding regime, magazine training began. On each of the first four days of training each rat received a 30 minute session of magazine training during which pellets were delivered on a variable interval (VI) 30 s schedule. Subsequently rats were then given four daily days of lever press training where each lever press resulted in the delivery of a food pellet (continuous reinforcement or CRF training). These sessions lasted until either 30 minutes expired or 30 food pellets were delivered. Rats which did not lever press during these initial sessions received additional training. Over the next three days the session length was increased from 30 to 60 minutes and the schedule on which the pellets were delivered was changed from a variable interval 30 s schedule (VI 30 s) to a VI 90 s schedule. On the VI 90 s schedule pellets could be earned by pressing the lever, on average, once every 90 s. The houselight was off throughout all of the training sessions.

CER training sessions were approximately 60 minutes long and usually conducted on 5 days per week. During these sessions, food pellets continued to be available on an VI 90 s schedule as before. However, the rats were now presented with two two-minute long periods of houselight illumination. The first light presentation occurred approximately 20 minutes after the start of the session (range 15-25 minutes) and the second light presentation occurred approximately 20 minutes after the first light presentation (range 15-25 minutes). Light presentations were occasionally followed by a 0.5 second 0.4 mA footshock. In a given session, footshock could follow neither, one, or both of the light presentations. On average rats would receive one shock presentation per session. The rate of lever-pressing during each of the light presentations (the ‘during’ or ‘dur’ period) and the rate of lever-pressing in the two minute interval preceding each light presentation (the ‘pre’ period) was recorded. These response rates were used to calculate suppression ratios using the following formula:

$$\frac{Response rate during}{( Response rate during ) + ( response rate pre )}$$

A suppression ratio (SR) of 0 indicates that the light has evoked conditioned fear and has completely suppressed lever pressing, whereas a SR of 0.5 indicates the response rate is unchanged by the light presentation: the complete absence of fear.

Once a reliable SR was observed, occasional test sessions occurred. Test sessions were identical to training session except that footshock was never delivered following the first light presentation. Tests occurred no more frequently than once per week and a baseline training session was always carried out before each test session.

Prior to test sessions, the rats were semi-randomly assigned to groups such that the groups were matched for the rate of lever pressing and the level of conditioned suppression displayed during baseline training. Drug treatment in each study was orthogonal to drug treatment which may have been administered to the animals in the previous study. Rats which had suppression ratios of greater than 0.15 or a mean pre score of less than 2 lever presses during the previous baseline session were excluded from the experiment.

The data were analysed with a mixed within-subject (trials) and between-subjects (drug treatment) analysis of variance (ANOVA). The ANOVA was conducted separately on the pre and during light response rates and on the suppression ratios. Significant interactions were followed by one-way analysis of variance on the data collected for each trial. Significant group effects were analysed using post-hoc Dunnett t-tests. The data for each experiment are presented collapsed across the two test trials.

Suppression ratios could not be calculated if no lever presses occurred during the pre light period. Thus, animals which failed to respond during either the first or the second light presentation were excluded from the analysis of suppression ratios.

The research described below was conducted within the guidelines of the Home Office regulations as outlined in the Animal (Scientific Procedures) Act, 1986.

Drugs

Sabcomeline ([R-(Z)]-α-(methoxyimino)-α-(1-azabicyclo[2.2.2]oct-3-yl) acetonitrile monohydrochloride, 0.01. 0.03, 0.1 and 0.3 mg/kg, sc) was dissolved in saline (supplied by Sigma-Aldrich Company Ltd., Fancy Road, Poole, Dorset, BH12 4QH; batch: S7653, 90322004) and administered at 2 ml/kg 10 min pre-test. To verify that the task was sensitive to clinically efficacious anxiolytics within each test, chlordiazepoxide hydrochloride (CDP) (10 mg/kg, po) (supplied by Sigma-Aldrich Company Ltd., Fancy Road, Poole, Dorset, BH12 4QH; batch: C2517, 94H1023) was dissolved in 1% methyl cellulose (supplied by Sigma-Aldrich Company Ltd., Fancy Road, Poole, Dorset, BH12 4QH; batch: M0262. 105H0489) and administered 2 ml/kg 40 min pre-test.

Results

Animals treated with 0.1 and 0.3 mg/kg sabcomeline made no pre responses on either trial one or two. This precluded the generation of a SR value and, therefore, these doses were excluded from statistical analysis. The ANOVA revealed a main effect of drug treatment on pre period responding (F[3,26]=9.4, p<0.01) but not treatment x trial interaction (F<1). A post hoc Dunnett's t test demonstrated that this effect was a consequence of a significant increase in pre responding after chlordiazepoxide relative to controls (Table 1). Analysis also supported a main effect of drug treatment on dur responses (F[3,26]=7.6, p<0.01) but no treatment x trial interaction (F[3,26]=1.1). A post hoc Dunnett's t test revealed that the main effect was the result of a significant increase in dur responding following CDP treatment as compared to vehicle (Table 2). Similarly, there was a significant main effect of drug treatment on SR (F[3.26]=9.6, p<0.01) but no treatment x trial interaction (F[3.26]=2.47). A Dunnett's t test revealed that this effect was mediated by a significant increase in SR after 0.03 mg/kg sabcomeline and CDP compared to vehicle. Thus, the effect on SR following administration of sabcomeline (0.03 mg/kg) was similar to that of CDP, a compound known to possess anxiolytic properties.

TABLE 1
Effect of sabcomeline and CDP on pre and dur response rates

Rate of responding

| | Dose | per minute ±SEM | |
|---|---|---|---|
| | (mg/kg) | “Pre” period | “Dur” period |
| Vehicle | — | 14.1 ± 1.1 | 2.3 ± 0.7 |
| Sabeomeline | 0.01 | 12.4 ± 1.6 | 3.0 ± 1.2 |
| Sabeomeline | 0.03 | 6.9 ± 1.5 | 6.8 ± 1.9 |
| Chlordiazepoxide | 10.0 | 21.8 ± 2.9* | 13.7 ± 3.1* |

TABLE 2
Effect of sabcomeline and CDP on suppression ratio

| | Dose | Suppression Ratio |
|---|---|---|
| | (mg/kg) | (± SEM) |
| Vehicle | — | 0.14 ± 0.04 |
| Sabcomeline | 0.01 | 0.15 ± 0.04 |
| Sabcomeline | 0.03 | 0.45 ± 0.06* |
| Chlordiazepoxide | 10.0 | 0.37 ± 0.05* |

*Significantly different relative to the vehicle-treated control group

Discussion

This is the first study to demonstrate an anxiolytic like profile for the muscarinic agonist sabcomeline in the CER procedure. Sabcomeline (0.03 mg/kg) significantly increased suppression ratios indicating an anxiolytic-like profile. Sabcomeline non-significantly increased responding during the conditioned stimulus period (dur) although the magnitude was smaller than that seen following CDP administration. Sabcomeline also markedly reduced baseline responding (pre), although this was not statistically reliable.

It is clear that the non-significant reduction in baseline responding following sabcomeline administration facilitated an increase in suppression ratios. However, it should be recalled that the same dose also increased responding, albeit non-significantly, in the conditioned stimulus period as compared to controls. Such a profile is not commensurate with a general non-specific reduction in lever pressing and is similar to that of the clinically efficacious anxiolytic diazepam at doses >2.5 mg/kg in the same model (Stanhope & Dourish Psychopharmacology. 128, 293-303, 1996). These data are consistent with sabcomeline evoking an anxiolytic-like profile in this procedure.

These results suggest that Compound (I) may have efficacy in the treatment of anxiety.

1. The use of [R-(Z)]-α-(methoxyimino)-α-(1-azabicyclo[2.2.2]oct-3-yl)acetonitrile or a pharmaceutically acceptable salt thereof for the treatment of anxiety.

2. The use according to claim 1 for the treatment of anxiety in patients other than those with Alzheimers's Disease.

3. A method for the treatment of anxiety comprising administering to the patient an effective, non-toxic amount of of [R-(Z)]-α-(methoxyimino)-α-(1-azabicyclo[2.2.2]oct-3-yl)acetonitrile or a pharmaceutically acceptable salt thereof.

4. A method according to claim 3, for the treatment of anxiety in patients other than those with Alzheimers's Disease.

5. A pharmaceutical composition, which comprises [R-(Z)]-α-(methoxyimino)-α-(1-azabicyclo[2.2.2]oct-3-yl)acetonitrile or a pharmaceutically acceptable salt thereof, and a pharmaceutically acceptable carrier for use in the treatment of anxiety.

6. A pharmaceutical composition, for the treatment of anxiety in patients other than those with Alzheimers's Disease

7. A use, method or composition according to any preceding claim wherein the pharmaceutically acceptable salt is the monohydrochloride.
